(19) United States
(12) Patent Application Publication
(10) Pub. No.: US 2012/0016763 A1
(43) Pub. Date: Jan. 19, 2012

(54) METHOD OF PROVIDING PRESCRIPTION SAFETY EYEWEAR

(76) Inventor: Bradley Kirschner, Homewood, IL (US)

(21) Appl. No.: 13/183,532

(22) Filed: Jul. 15, 2011

Related U.S. Application Data
(60) Provisional application No. 61/364,895, filed on Jul. 16, 2010.
(60) Provisional application No. 61/372,559, filed on Aug. 11, 2010.

(51) Int. Cl. G06Q 30/00; G06F 3/048; A61B 3/11

(52) U.S. Cl. 705/26.5; 351/204; 715/835; 351/246

(57) ABSTRACT

A system and an apparatus for an employer to provide safety prescription eyewear to employees is described. The steps of the preferred embodiment comprise providing a kiosk at or near a place of employment, allowing employees to access the kiosk virtually to try on various models of frames and to submit prescription information, and causing the prescription safety eyewear to be manufactured and shipped. Employee benefits are handled at the kiosk as are all options available relating to, for examples, the selected frame, the prescription lenses, and the method of shipping. Payments in excess of the benefit are accounted for by payment at the kiosk or by payroll deduction.

### PRIORITY

This application claims priority from U.S. Provisional Patent Application No. 61/364,895, filed Jul. 16, 2010, and U.S. Provisional Patent Application No. 61/372,559, filed Aug. 11, 2010, the disclosures of which are incorporated herein.

### BACKGROUND OP THE INVENTION

This invention relates generally to the provision of prescription eyewear and more specifically to the provision of prescription safety eyewear to employees for use in the workplace.

Many workplaces require employees to wear safety glasses at the workplace. This requirement is generally a matter of worker safety but is also usually required by state and federal law. Workers who need corrective lenses have a choice of wearing prescription safety eyewear or wearing bulky, usually uncomfortable, safety goggles over their regular prescription eyewear. Many workers who need corrective lenses prefer to wear prescription safety eyewear for reasons of at least comfort and appearance. Accordingly, there is a need for prescription safety eyewear for use in the workplace.

Many employers, therefore, provide prescription safety eyewear as an employment benefit, paying for some or all of the cost of such eyewear. Conventionally, however, employees have to obtain their own prescription eyewear and submit the cost for reimbursement, which is inconvenient for both the employer and the employee. For example, administrative difficulties arise for the employer in tracking eligibility for benefits. Employees might resent having to spend personal time obtaining company-required equipment. One alternative is to have employees order from a catalog, which presents problems as to fit of the eyewear on the employee. Another alternative is to have an eyewear seller come to the place of employment for selection and fitting of prescription safety eyewear, which does not work well for employees who, for example, are assigned to a late shift or who are absent from the plant on vacation or on business on the day the seller is present at the place of employment.

The present invention is meant to address the problems of the prior art and to provide an ameliorative solution.

### BRIEF SUMMARY OF THE INVENTION

The invention comprises in general a system for an employer to provide safety prescription eyewear to employees. The steps of the preferred embodiment comprise providing a kiosk at or near a place of employment, allowing employees to access the kiosk virtually to try on various models of frames and to submit prescription information, and causing the prescription safety eyewear to be manufactured and shipped. Employee benefits are handled at the kiosk as are all options available relating to, for examples, the selected frame, the prescription lenses, and the method of shipping. Payments in excess of the benefit are accounted for by payment at the kiosk or by payroll deduction.

### BRIEF DESCRIPTION OF THE SEVERAL VIEWS OF THE DRAWINGS

The organization and manner of the structure and operation of the invention, together with further objects and advantages thereof, may best be understood by reference to the following description, taken in connection with the accompanying non-scale drawings, wherein like reference numerals identify like elements in which:

FIG. 1 is a block diagram of a kiosk of the preferred embodiment of the present invention.

FIG. 2A is a sample first screenshot seen by an employee accessing the kiosk of FIG. 1.

FIG. 2B is a sample log-in screenshot seen by an employee accessing the kiosk of FIG. 1.

FIG. 2C is a sample account confirmation screenshot seen by an employee accessing the kiosk of FIG. 1.

FIG. 2D is a sample photography screenshot seen by an employee accessing the kiosk of FIG. 1.

FIG. 2E is a sample screenshot showing an employee accessing the kiosk of FIG. 1.

FIG. 2F is a sample screenshot showing an image of the employee of FIG. 2E with the template alignment tool and pupil alignment tool superimposed thereon.

FIG. 2G is a magnified portion of a sample screenshot showing the image of FIG. 2F with the pupil alignment tool properly adjusted.

FIG. 2H is a sample screenshot showing the image of FIG. 2F with the template alignment tool properly adjusted.

FIG. 2I is a sample screenshot of the frame selection menu and displayed photograph showing a frame selected by the employee accessing the kiosk of FIG. 1.

FIG. 2J is a sample optometry prescription display screenshot seen by an employee accessing the kiosk of FIG. 1.

FIG. 2K is a sample scanner-prompt screenshot seen by an employee accessing the kiosk of FIG. 1.

FIG. 2L is a sample lens options screenshot seen by an employee accessing the kiosk of FIG. 1.

FIG. 2M is a sample lens material options screenshot seen by an employee accessing the kiosk of FIG. 1.

FIG. 2N is another lens recommendations screenshot seen by an employee accessing the kiosk of FIG. 1.

FIG. 2O is yet another sample further options screenshot seen by an employee accessing the kiosk of FIG. 1.

FIG. 2P is a confirmation of selection screenshot seen by an employee accessing the kiosk of FIG. 1.

FIG. 2Q is a confirmation of employer benefits screenshot seen by an employee accessing the kiosk of FIG. 1.

FIG. 2R is a sample final payment screenshot seen by an employee accessing the kiosk of FIG. 1.

### DETAILED DESCRIPTION OF THE PREFERRED EMBODIMENTS OF THE INVENTION

While the invention may be susceptible to embodiment in different forms, there is shown in the drawings, and herein will be described in detail, specific embodiments with the understanding that the present disclosure is to be considered an exemplification of the principles of the invention, and is not intended to limit the invention to that as illustrated and described herein.

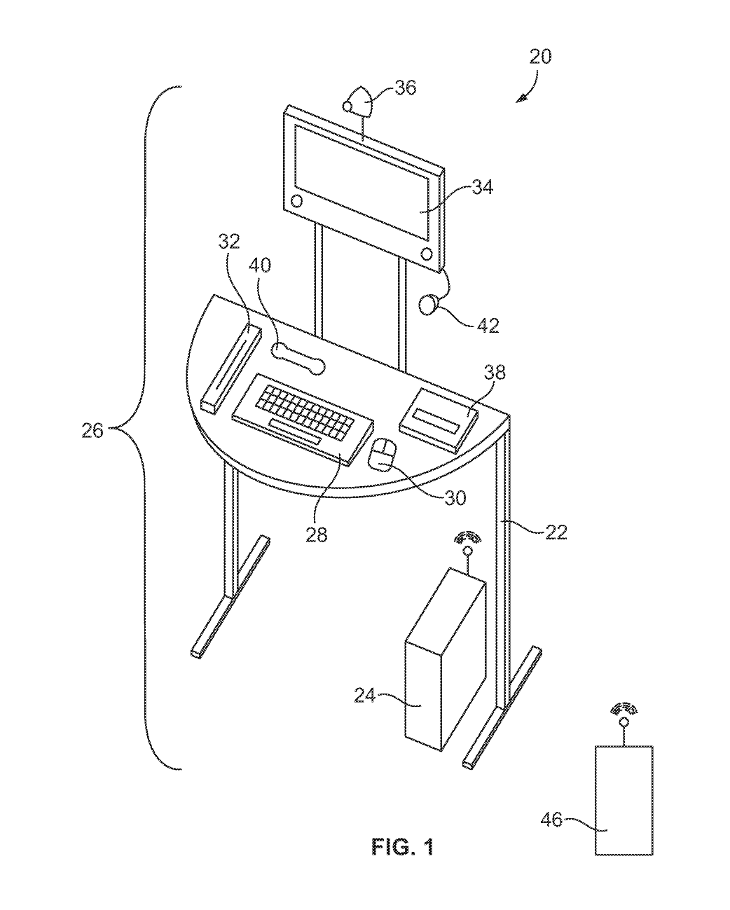

The system of the preferred embodiment of the present invention comprises a kiosk 20, shown in diagrammatic form in FIG. 1. Kiosk 20 comprises a frame 22, a central processing unit 24, and computer peripherals 26, preferably comprising a keyboard 28, a mouse 30, a scanner 32, a display 34, a camera 36, and a printer 38, all of which are set on, mounted on, connected to, or nearby frame 22. The computer peripherals 26 are coupled to CPU 24 wirelessly, as shown, or by hard wires. CPU 24 is connected to the Internet by a wireless connection, as shown, or by hard wire. The Internet connection is preferably a broad-band connection such as DSL or T1. Alternatively, CPU 24 can connect to the employer's intranet. Also provided with kiosk 20 is a fitting tool, template 40, which is used for fitting, as will be described in more detail below.

Kiosk 20 can also have microphone 42 for voice-activated commands, in which case CPU 24 is programmed with commercially-available voice-activation software. The following description will be for entry of commands using keyboard 28 and mouse 30, but the same results can be used by programming CPU 24 to recognize voice commands received through microphone 42.

Mouse 30 can be replaced by a touchpad, joystick, or other peripheral. Display 34 can be a touch screen display, in which case mouse 30 is not required.

Display 34 is preferably a flat-screen LCD with internal speakers 42. Display 34 can be other types, such as a CRT screen, another type of flat screen, or other visual display, and kiosk 20 can have speakers external to display 34. The following description will be for an embodiment using internal speakers for voice commands and prompts, but, alternatively, kiosk 20 can operate without speakers and display commands and prompts in writing only.

The preferred embodiment applies to safety eyewear for company employees. Accordingly, the following description will be for an embodiment in which a kiosk is placed at a placed of employment or affiliated facility. The invention can also be implemented by placing the kiosk in a retail store in which the user purchases his or her own safety eyewear, either for work use or for personal use.

In the preferred embodiment, an employee who wishes to obtain prescription safety eyewear begins by accessing kiosk 20. Kiosk 20 is meant to be located at an employer's workplace where safety eyewear required, such as a manufacturing plant. Kiosk 20 can be located conveniently for the employees, such as in or adjacent to locker rooms, break rooms, cafeterias, front offices, or at other convenient locations such as an employee credit union or a labor union office.

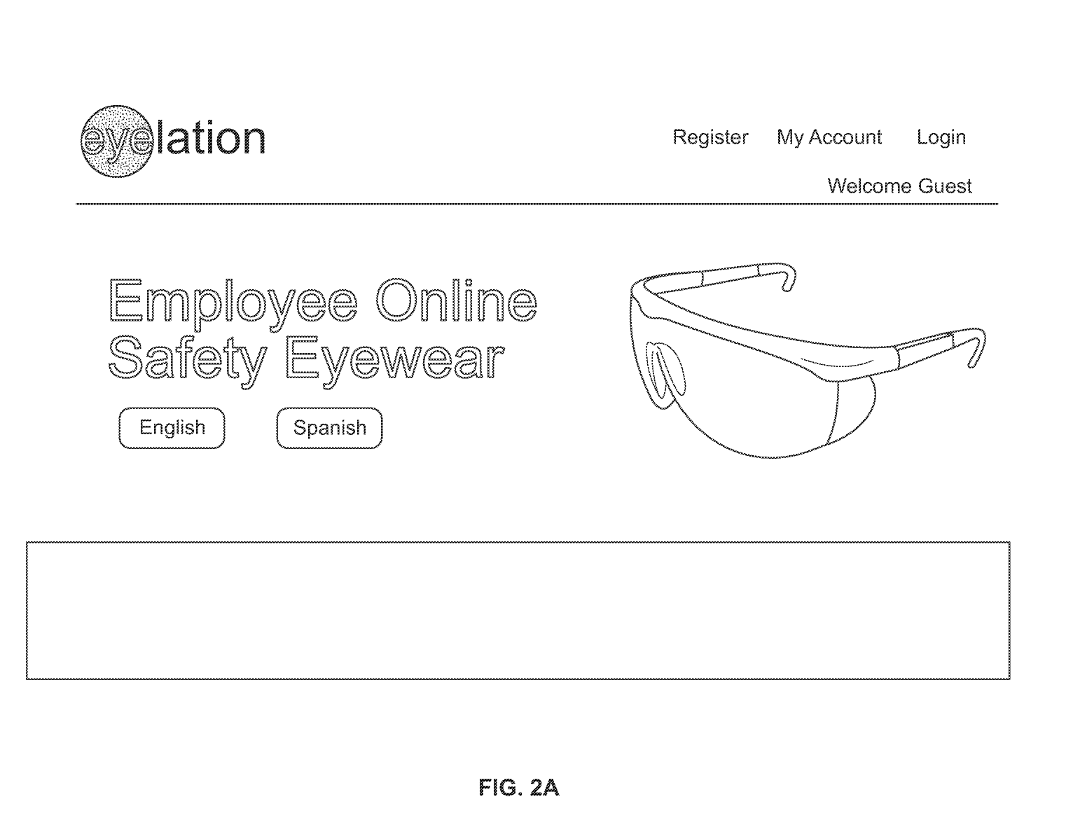

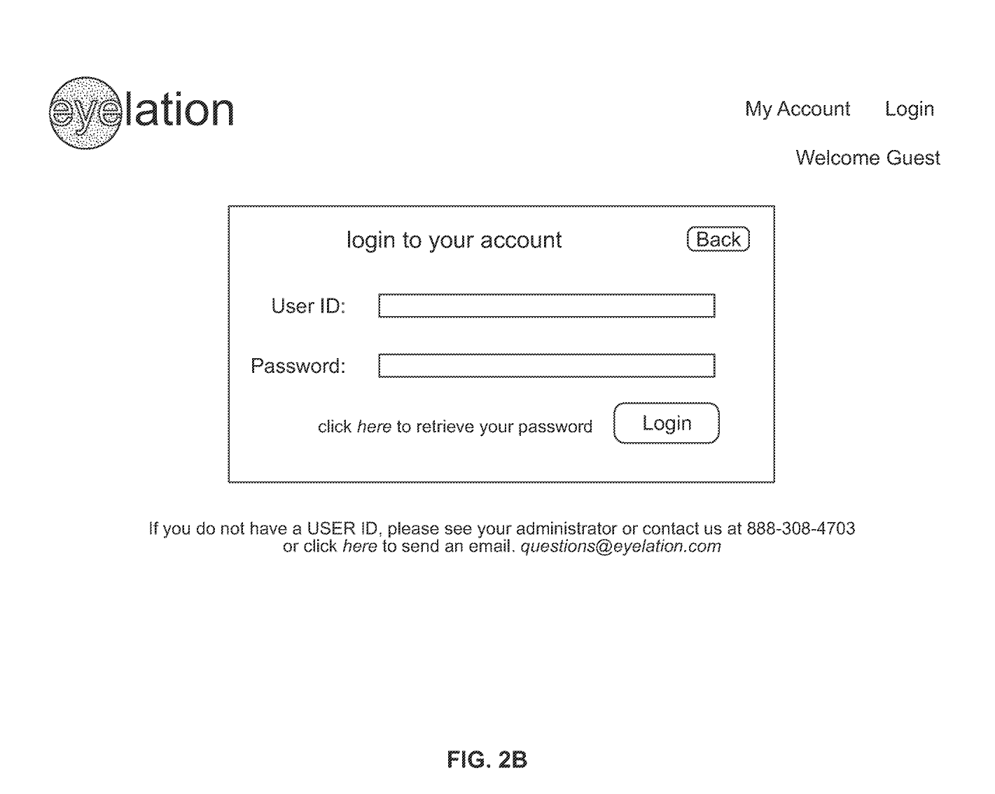

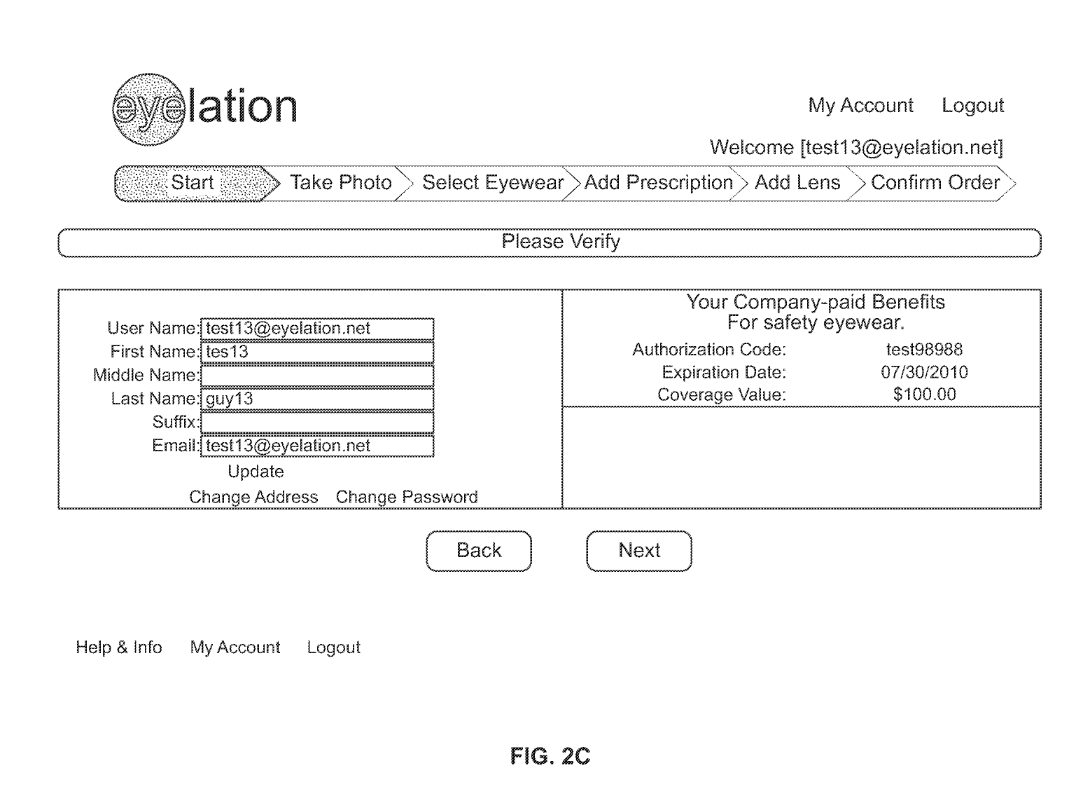

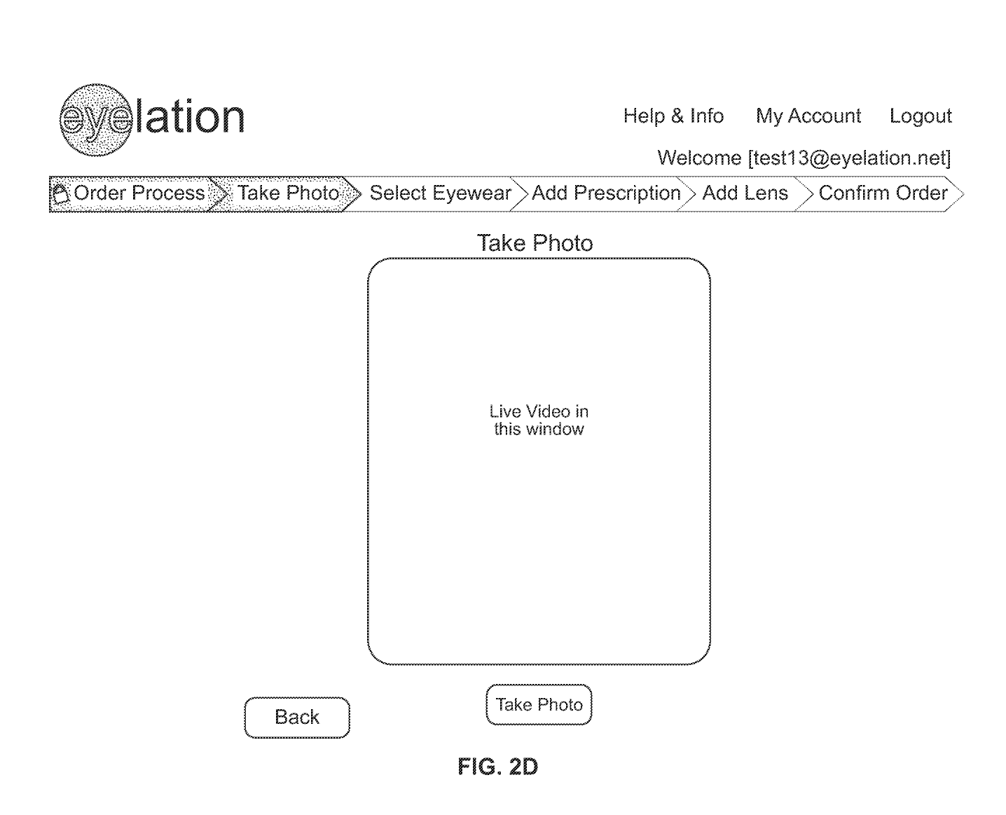

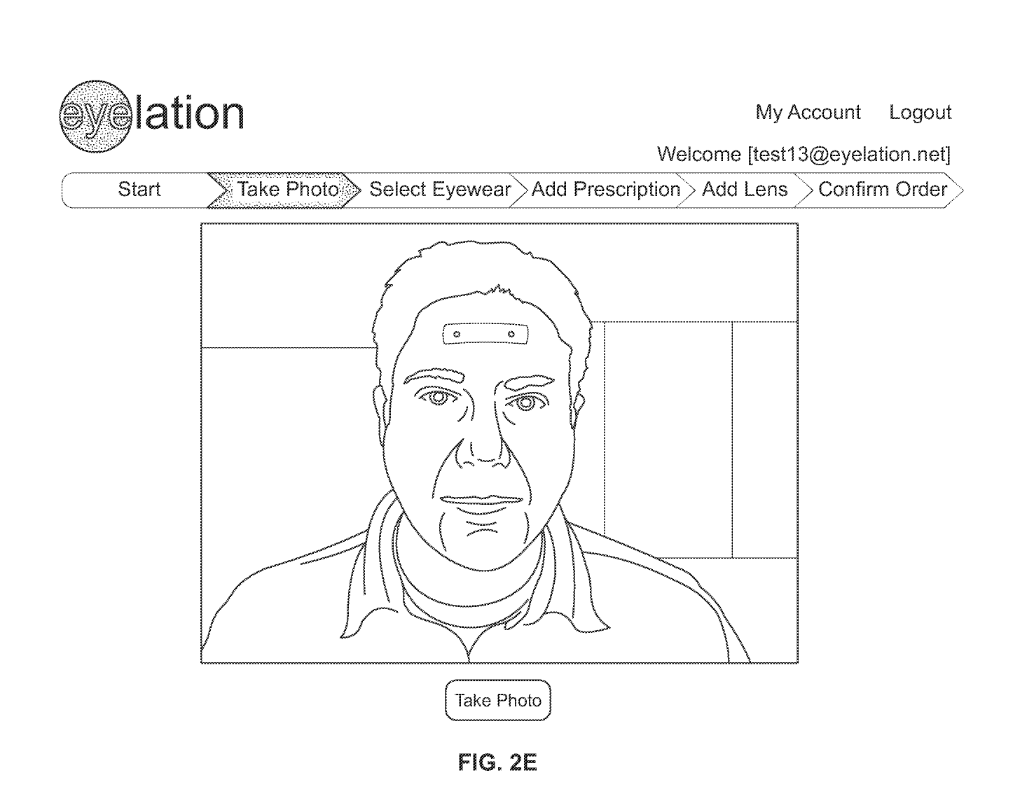

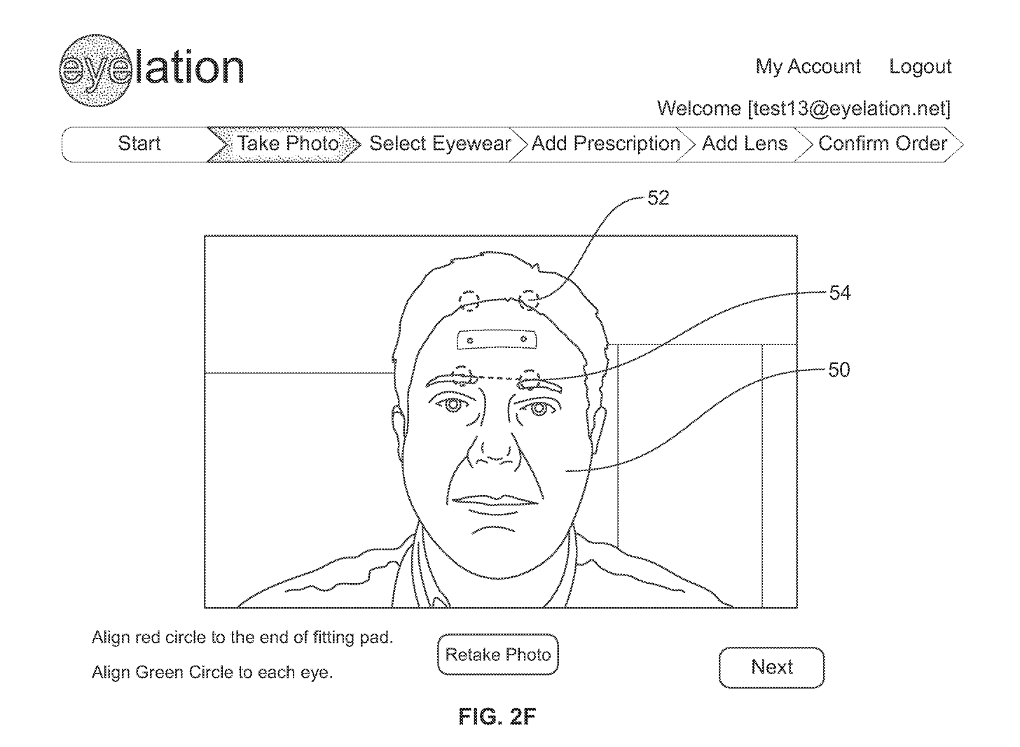

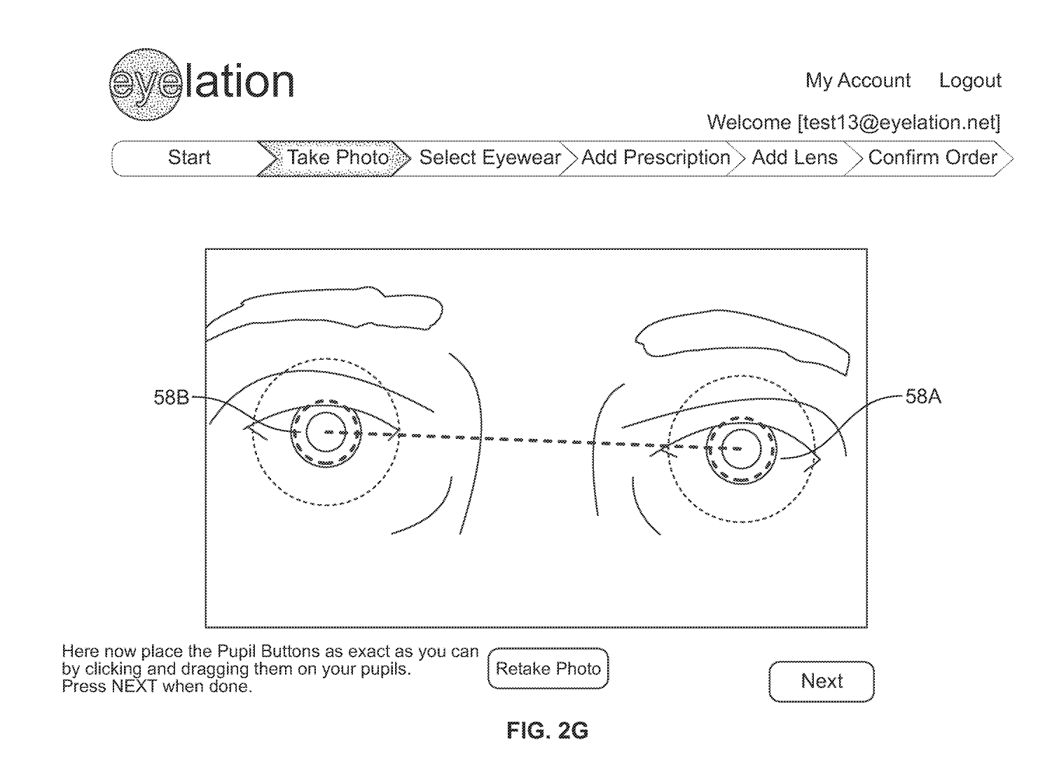

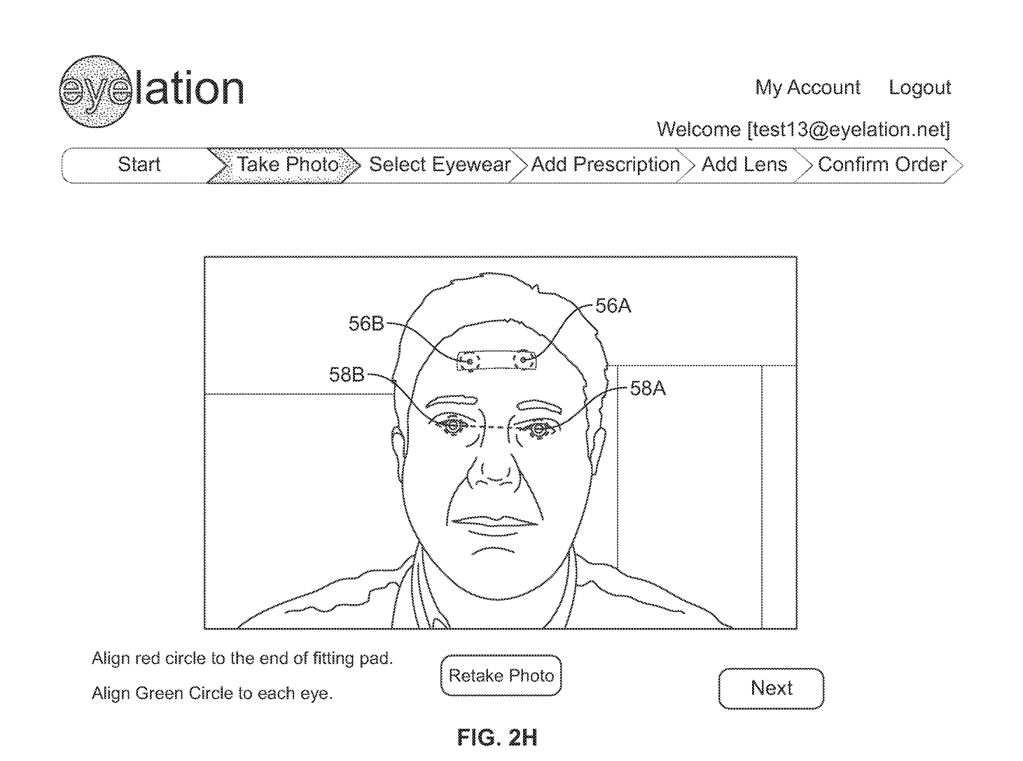

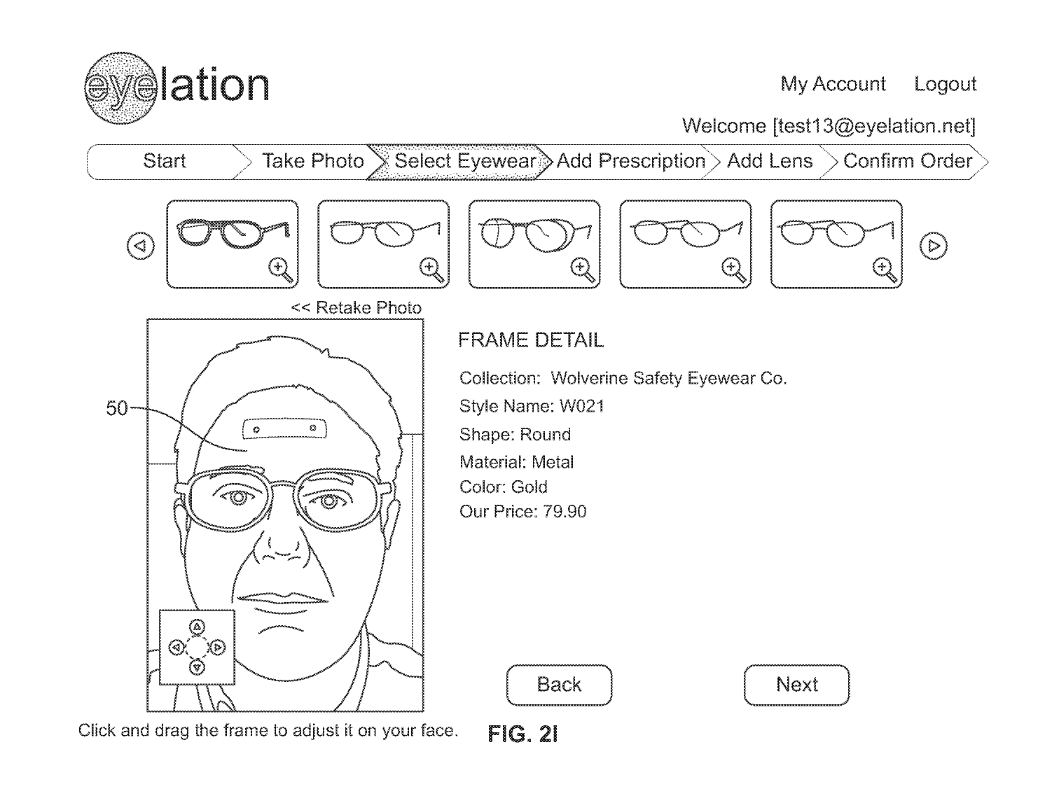

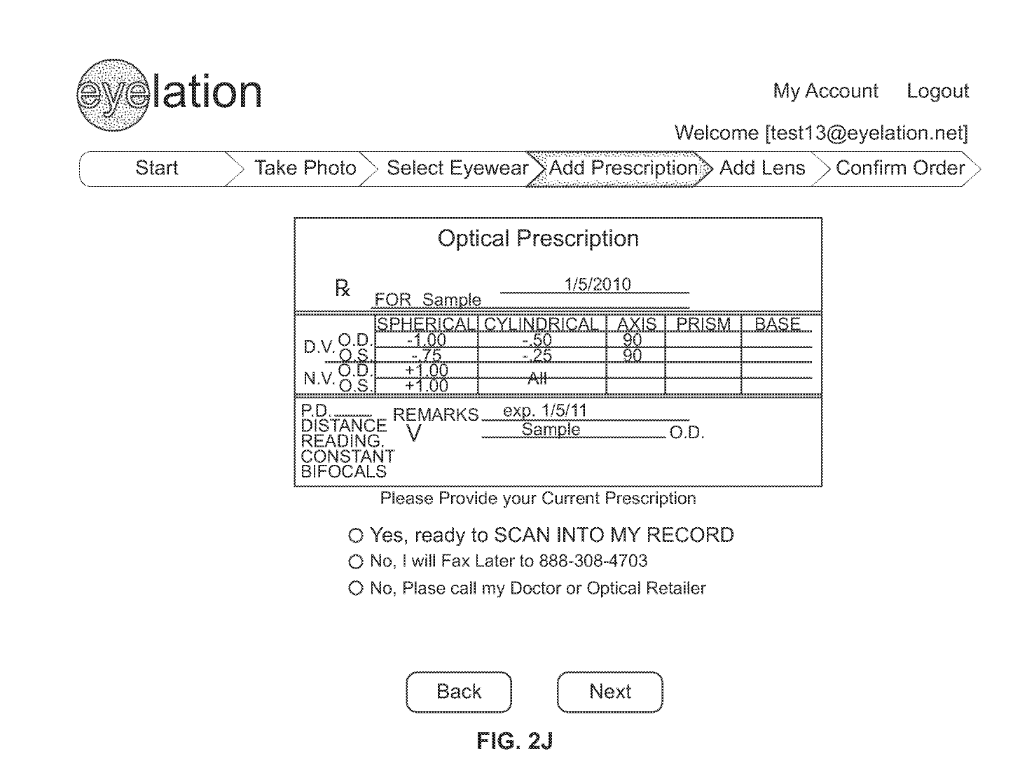

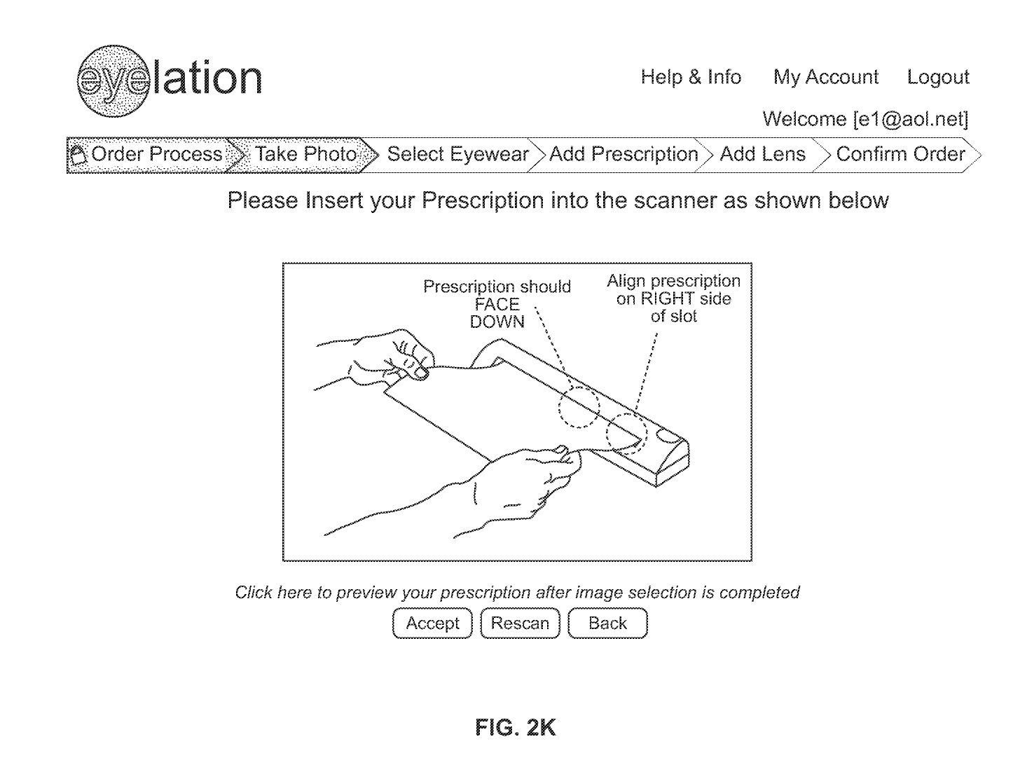

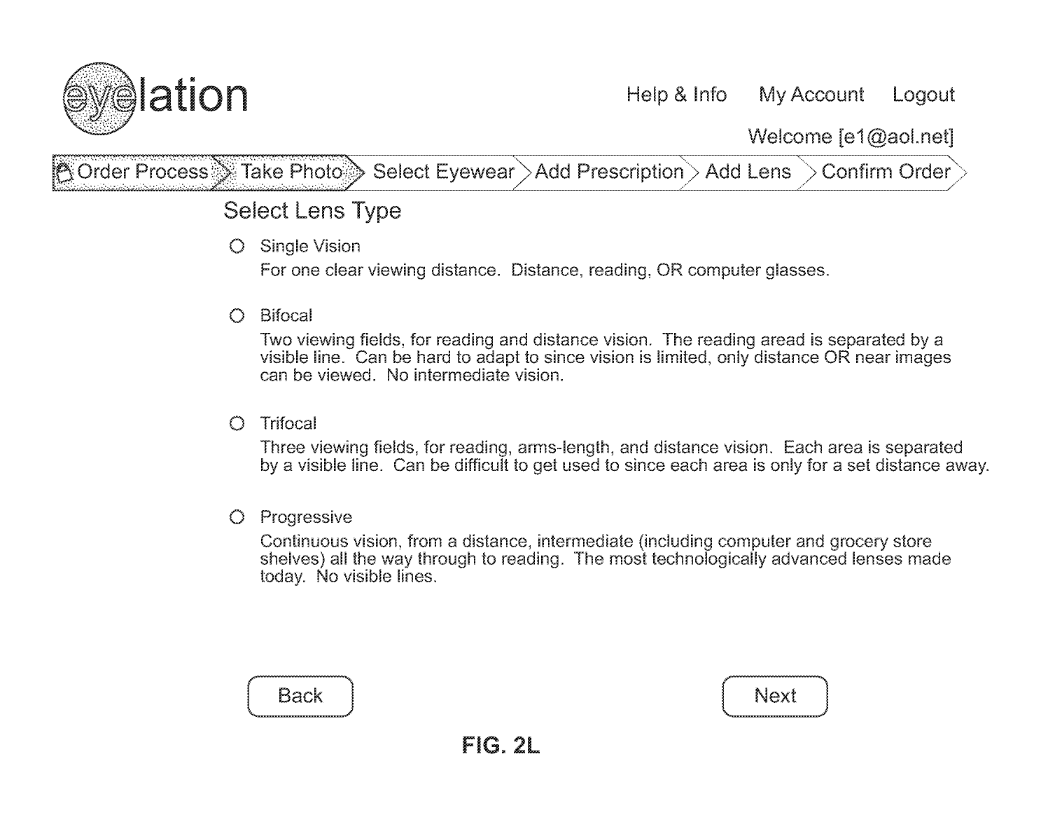

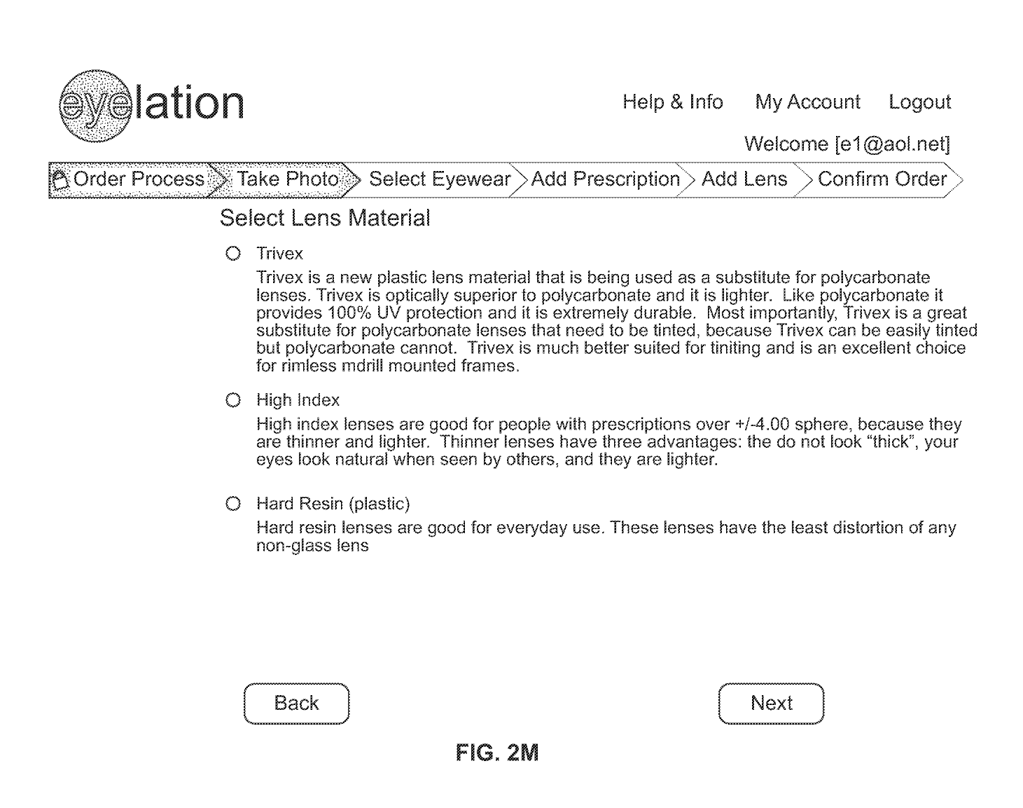

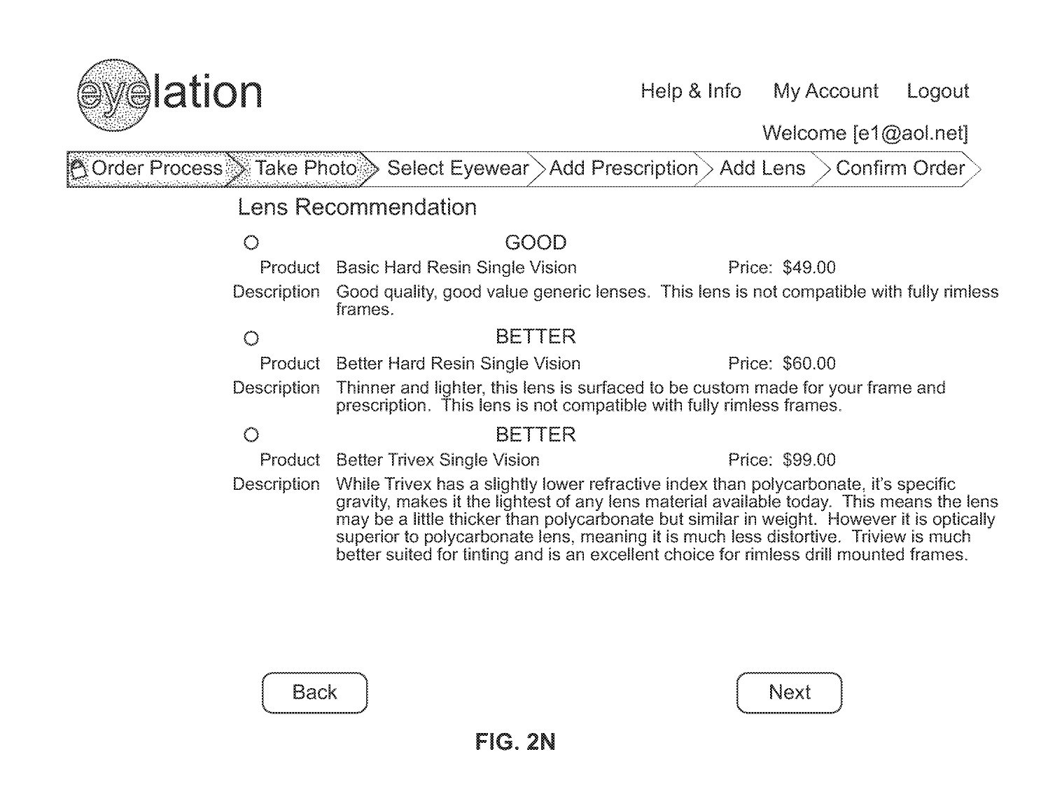

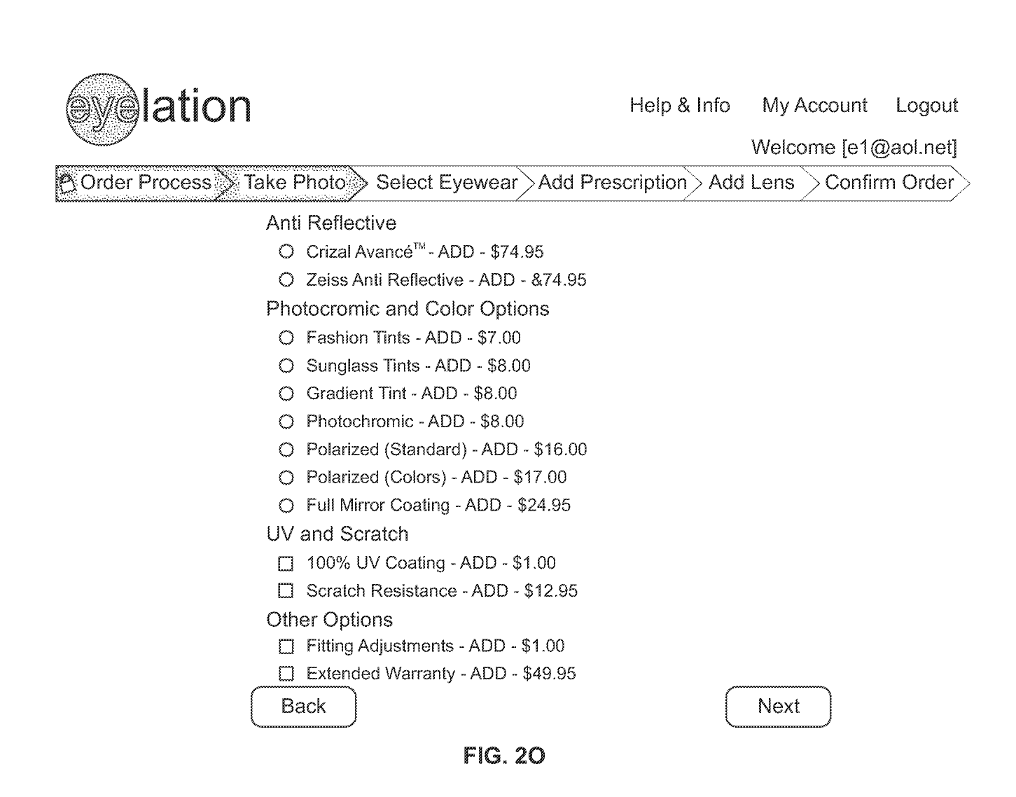

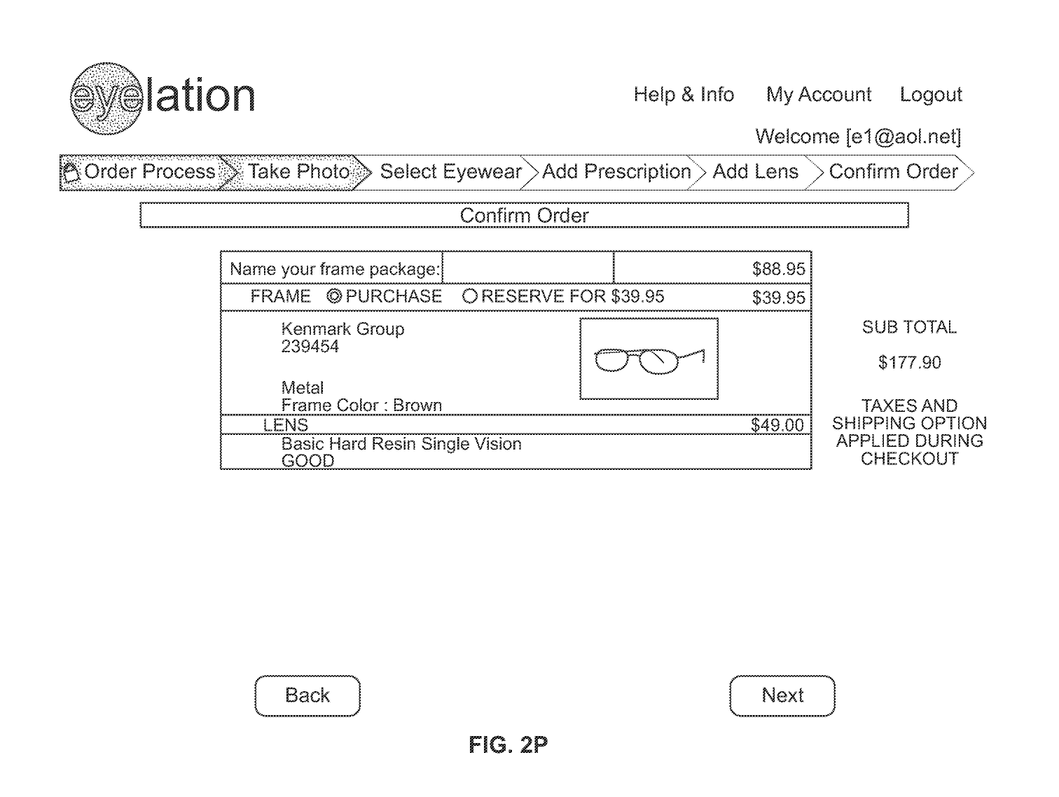

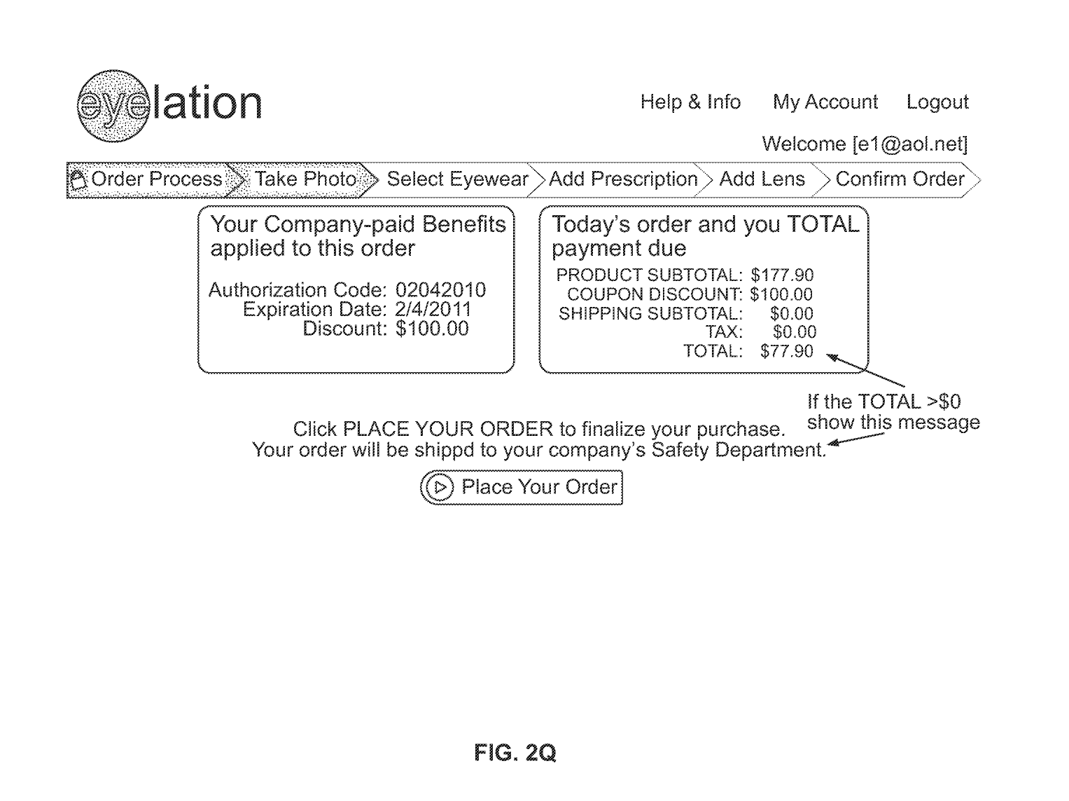

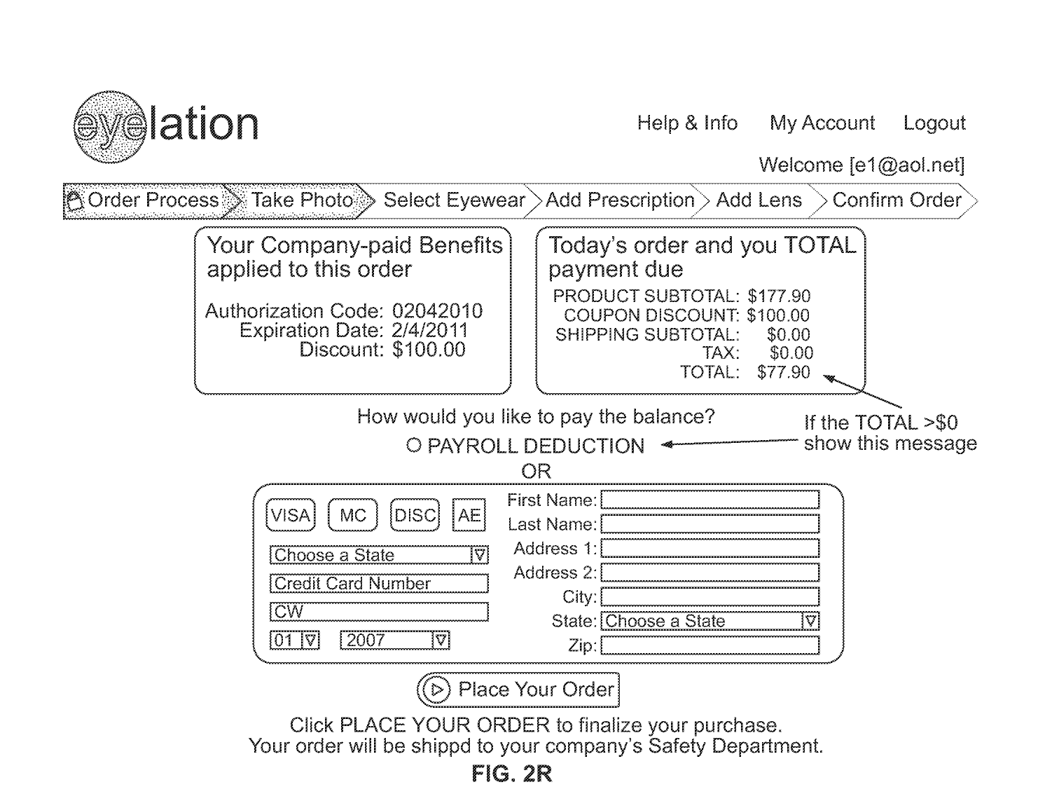

The employee initiates kiosk 20 by entering any key on keyboard 28 or by moving mouse 30. Alternatively, an on-off switch can be provided or a proximity switch can be used to start kiosk 20 upon the approach of a person. The first screenshot seen by the employee upon initiating kiosk 20 is shown in FIG. 2A, which is a general welcome screen and preferably has a choice of languages for selection by the employee where appropriate. Selection of a language brings up the screenshot shown in FIG. 2B, which is a conventional log-in screen. The employee then uses keyboard 28 to enter a company-issued identification name or number and a password. Kiosk 20 verifies the employee's identity and authority to access kiosk 20, either by comparison to data stored in CPU 24 or, more preferably, by comparison to data stored in a central server 46 that is accessed by CPU 24 over the Internet or local intranet. Other types of verification can be used, including, for examples, a magnetic card reader or MD reader for employee identification cards. The employee in these situations can swipe an employee identification card or a benefits card through a magnetic card reader, or holds the employee identification card or benefits card near the RFID reader.

Upon recognition and authentication of the employee, kiosk 20 displays the screenshot shown in FIG. 2C, which confirms the employee's name, email address, employee identification number, amount of benefits available, and expiration of eligibility for the benefits. The employee confirms this information, or edits it if necessary and then confirms, by clicking a “Next” button with mouse 30.

The employee is then presented h the screenshot shown in FIG. 2D. A voice command advises the employee to affix template 40 to his or her forehead, to stand before camera 36 and look directly into the lens of camera 36. The employee's image is then shown on display 34, as shown in FIG. 2E. The employee takes a photograph by clicking the “Take Photo” icon, by entering any key, or by entering a specifically-named key, on keyboard 28.

CPU 24 receives digital data from camera 36 and creates an image 50 of the employee, preferably in jpeg format. Using conventional digital recognition software, CPU 24 rotates image 50, if necessary, and automatically provides other digital image processing if necessary to correct for improper lighting or other imaging issues.

Kiosk 20 now displays image 50 on display 34 on the screenshot shown in FIG. 2F, with a template alignment tool 52 and pupil alignment tool 54 superimposed on image 50. Both template alignment tool 52 and pupil alignment tool 54 are software-implemented tools programmed into CPU 24 to superimpose images on image 50. As shown in FIG. 2F, tools 52, 54 are not properly aligned.

Kiosk 20 instructs the employee to use mouse 30 to move pupil alignment tool 54 to place two circles 58a, 58b on the pupils of the employee's image 50, as shown in FIG. 2G. When the employee has done so and confirms by clicking “Next”, kiosk 20 instructs the employee to use mouse 30 to move the two circles 56a, 56b of template alignment tool 52 to opposite edges of template 40 as it appears in image 50 on the forehead of the employee. As shown in FIG. 2H, both pupil alignment tool 54 and template alignment tool 52 are properly aligned on the image of the employee's face. The employee clicks the “Next” icon when finished, or opts for a different picture.

In another aspect of the invention, CPU 24 is programmed with autodetect software. CPU 24 detects the employee's eyes and detects template 40 on the image. In this aspect, the employee then confirms that CPU 24 has properly autodetected the pupils and template 40.

CPU 24, knowing the distance between the opposite edges of template 40, calculates the pupil distance between the employee's eyes, regardless of the distance between the employee's face and camera 36 at the time image 50 was captured.

The employee now selects a frame from a menu. As illustrated in FIG. 2I, the employee can navigate through the menu by clicking arrows on either side. Any other type of menu navigation procedure can be used. When the employee sees a desired frame, he or she clicks on the frame, whereby the selected frame is superimposed over image 50. Since CPU 24 has calculated the employee's pupil distance using the template alignment tool 52 and pupil alignment tool 54 procedure above, and therefore has information relating to the relative size of the employee's face in image 50, from template 40, CPU 24 automatically sizes the selected frame properly to fit on the employee's face in image 50. The employee is prompted to click and drag the selected frame to adjust its location on the image. Accordingly, the employee can see how he or she will look in the selected, properly-fitted frame, as shown in FIG. 2I.

If the employee is satisfied with the selected frame, the process continues. Otherwise, the employee can select a different frame and see how he or she will look in that different selection. The employee can continue until a final selection is made. If available under company benefit's policy, or if the employee chooses at his or he own cost, the employee can select more than one frame for purchase.

The employee can, at this point, choose to print image 50 with a selected frame shown thereon, and to save all data up to this point. The employee can then take printed image 50, or even several printed images 50 with different selected frames, for further consideration or to obtain opinions from others, before returning at a later time and picking up the process again at this point.

If the employee is satisfied with the selected frame, kiosk 20 now proceeds to the screenshot shown in FIG, 2J. The employee's last prescription, if available, is called from memory, either from the memory of CPU 24 or from central server 46, and is displayed. The employee is given the option to use the current prescription if it is recent, by clicking the “Next” icon. Kiosk check the age of the prescription and can be programmed to refuse to accept a prescription older than a predetermined age. The employee is also given the option to scan in a new prescription, even if the initial prescription is not expired, to fax a new prescription later, or to suggest contact with the employee's optician, optometrist, or ophthalmologist, as described in FIG. 2J.

If the employee has a new prescription or is a new employee, the next screenshot, shown in FIG. 2K, displays instructions on scanning the prescription using scanner 32. The employee then scans the prescription into scanner 32, which relays that information to CPU 24, and the employee's database is updated thereby.

Kiosk 20 confirms that the prescription was properly scanned and downloaded, and gives the employee an option to rescan if required. Otherwise, when the employee accepts that the prescription has been scanned, kiosk 20 proceeds to options available to the employee.

Some sample options are shown in the next several screens. The screenshot shown in FIG. 2L shows options for lens type. The screenshot shown in FIG. 2M shows options for lens material. The employee chooses whatever options are desired from those screens and navigates to the next screen by clicking “Next.” The following screenshot, shown in FIG. 2N, provides recommendations to the employee as to the options available, and permits the employee to go back to the screenshots shown in FIGS. 2L and 2M to re-select options.

Additional features, such as anti-reflective coating, color, or anti-scratch coatings are presented in the screenshot shown in FIG. 2O. The employee selects whatever additional features are desired and proceeds to the next screenshot, shown in FIG. 2P. Here, the total package is summarized and the final cost is given. The employee is given the option of saving for later, or completing the transaction, or starting over.

If the employee chooses to complete the transaction, the screenshot shown in FIG. 2Q is shown, summarizing the order, applying the benefits, if any, and asking the employee to complete the transaction. If the employee chooses to complete the transaction, the final screenshot, shown in FIG. 2R, is shown, with the final details. If a balance is due, such as if the employee went over the employer's benefit, an option is given to pay by credit card or by payroll deduction (if available). Printer 38 will print a confirmation for the employee for record-keeping purposes, such as tax deduction, if available. Alternatively, kiosk 20 will email the confirmation to the employee, using a stored email address or by prompting for entry of an email address.

After completion of the transaction, kiosk 20 transmits to central server 46 the details of the transaction. These details include the selected frame, selected options for the selected frame, the employee's prescription, the selected lens, selected options for the selected lens, the financial details, such as cost of the frame, lenses, and selected options, less the employer's benefit, and any discounts that may apply.

In some cases, the employee has not submitted a current prescription. The operator or its agent or contractor then must locate the telefacsimile sent by the employee, if that option was used, and manually enter the prescription information into central server 46.

In other cases, the employee chose the option of requiring the operator to contact the optician, optometrist, or ophthalmologist. In these situations, the operator or its agent or contractor must call for the prescription information and manually enter it, or obtain a telefacsimile from the optician, optometrist, or ophthalmologist and manually enter the prescription information from that document.

The operator of kiosk 20 next prepares the employee's selected prescription safety eyewear, either in-house or using one or more contractors. The operator or a designated contractor either has the selected frame, in the correct size and with the selected options, or purchases the selected frame from in the correct size and with the selected options from a manufacturer or broker, The operator causes the lenses to be prepared, either in-house or using a suitable contractor, to meet the employee's prescription and selected options. The operator then causes the lenses to be attached to the frame, again either in-house or using a suitable outside contractor, and the now-assembled prescription safety eyewear is shipped.

In the illustrated option, as shown in FIG. 2R, the final eyewear is shipped to the employer's place of business, such as the Safety Department. The Safety Department has the option, if the employer desires, of verifying that the selected frame and lenses are appropriate and meet government and industry standards, and then distributes the eyewear to the employee. This step may be eliminated if the employer accepts that all frames and lenses supplied by the operator are appropriate. Additionally, the employer can agree that the eyewear will be shipped directly to the employee. In that case, an additional screenshot is used in kiosk 20 to select shipping options, such as overnight, air freight, or standard, for any of which additional charges may be required.

A benefit of the method of the preferred embodiment to the employee is that the kiosk is always available and is located at the workplace or at some other location convenient to all employees. The employee therefore does not have to shop for eyewear on his or her own time and does not have to come to work at an inconvenient or unscheduled time. The employee can be assured that whatever eyewear is selected, it will meet company requirements, as the employer will require the operator to limit frame selection, lens selection, and options to those meeting company and/or industry and governmental standards. The employee will also have benefits automatically applied, without having to go through an arduous and possibly slow reimbursement procedure. The employee can also easily check what benefits are available and if a new benefit term has been reached.

The employer using the system of the preferred embodiment can be assured that employees will only purchase eyewear meeting company, industry, and governmental standards. The employer therefore can forego the need to check on an individual employee's eyewear purchase to be sure the eyewear is compliant. (The employer can check anyway if it chooses.) The employer can be assured that all personnel who are required to wear safety eyewear and who need prescription safety eyewear are obtaining properly-fitted, safety-standard-compliant equipment and are able to do so in a convenient manner. The employer also knows when any particular employee has ordered new eye d can take steps to make sure all employees are keeping their safety equipment current and in good repair.

The employer also has benefits automatically applied to its employees' purchase of safety prescription eyewear, saving the trouble of processing numerous hand-written reimbursement requests and allowing for easier, more transparent accounting of costs in this area.

Please note that the system as described can also be used for non-prescription eyewear. The software is merely modified to provide that the employee can skip the step of submitting a prescription.

The method of the preferred embodiment of the present invention comprises the following steps, not all of which are necessary to the invention and which need not necessarily be performed in the following order except where required by logic:
Step 101 The employee obtains a prescription for eyeglasses from an ophthalmologist, optometrist, r other health-care provider.
Step 102 The employee accesses a kiosk at or near the workplace or other convenient location by logging on, preferably with a user identification and password.
Step 103 The kiosk displays the authorization code, expiration date, and amount of benefits available on the display screen.
Step 104 The employee places a template on his or her forehead.
Step 105 The employee aligns his face with the camera of the computer screen and checks for proper alignment.
Step 106 The kiosk verifies proper alignment and advises the employee to adjust, if necessary.
Step 107 The employee causes the kiosk to photograph the employee's face by clicking a button.
Step 108 The kiosk corrects rotation of the employee's face in photograph, if necessary, and displays the corrected photograph on the display with the template measuring tool and the pupil measuring tool.
Step 109 The employee adjusts the template measuring tool to match the template displayed in the photograph.
Step 110 The employee adjusts the pupil measuring tool to match the pupils displayed in the photograph.
Step 111 The employee accepts the adjustment of the photograph or re-adjusts the template measuring tool or the pupil measuring tool, or chooses to take a new photograph.
Step 112 The employee selects frames from a menu and clicks on a selected frame.
Step 113 The kiosk displays the details of the selected frame, such as brand, material, color, shape, and price.
Step 114 The kiosk displays options available for the selected frame, if any, such as color.
Step 115 The kiosk displays the selected frame on the photograph.
Step 116 The employee accepts the selected frame, or changes the options, if any, or selects a different frame from the menu.
Step 117 The employee may save a selected frame at this point and return later.
Step 118 When the employee has accepted a selected frame, the kiosk prompts the employee to enter the prescription information, preferably by scanner.
Step 119 The employee enters the prescription information, preferably by inserting a current and valid prescription from his or her doctor into the scanner.
Step 120 kiosk displays the scanned prescription form and prompts the employee to accept the scan or to rescan.
Step 121 The kiosk displays a menu of lens options, if available.
Step 122 The employee selects lens options, if available.
Step 123 The kiosk displays prices based on the employee's selection of options.
Step 124 The kiosk displays recommendations for unselected options, if appropriate.
Step 125 The employee confirms the original selection or selects other options.
Step 126 Upon confirmation, the kiosk displays the final product and total cost.
Step 127 The employee selects shipping options, if available (for examples, overnight or standard freight), and location to be shipped (for examples, home or place of employment).
Step 128 The kiosk calculates the employer benefit, if available, applies that benefit to the total cost, applies a discount, if applicable, applies a shipping charge, if applicable, computes taxes due, if any, and displays a net cost.
Step 129 The employee confirms the order.
Step 130 if money is due in excess of the employer's benefit, if any, the kiosk prompts the employee for payment, such as by payroll deduction or credit card.
Step 131 The kiosk provider purchases the selected frame or picks the selected frame from previously-purchased stock, causes the lenses to be manufactured and applied to the selected frame, and causes the assembled eyewear to be Shipped to the location previously selected.

While preferred embodiments of the present invention are shown and described, it is envisioned that those skilled in the art may devise various modifications of the present invention without departing from the spirit and scope of the appended claims.

I claim:

1. A method for providing a prescription safety eyewear, said method comprising:
creating an image of a face, on which face a template has been placed;
displaying the image;
displaying a template alignment tool and a pupil alignment tool on the image;
receiving an adjustment of the pupil alignment tool;
determining a pupil distance of the face;
displaying a menu comprising images of a plurality of frames;
receiving a selection of a one of said plurality of frames;
sizing the selection to fit the face;
displaying the sized selection on the image;
receiving a prescription information; and
causing a prescription safety eyewear to be manufactured with the pupil distance and the selection.

2. The method of claim 1, further comprising the step of aligning the image and receiving a verification of a proper alignment of the image.

3. The method of claim 1, whereby the receiving a prescription information step comprises at least one of recalling the prescription information from a database and receiving a scanned image of a prescription form displaying the prescription information.

4. The method of claim 1, further comprising displaying a plurality of lens options and receiving a selection of a one of the plurality of lens options.

5. The method of claim 1, further comprising displaying a plurality of shipping options and receiving a selection of a one of the plurality of shipping options.

6. The method of claim 1, further comprising the step of determining an amount of benefits available.

7. The method of claim 6, further comprising the step of applying the amount to a cost of the prescription safety eyewear.

8. The method of claim 7, further comprising the step of receiving a payment for an excess of the cost over the amount.

9. The method of claim 1, further comprising the step of causing the prescription safety eyewear to be shipped.

10. The method of claim 1, further comprising the steps of receiving log-in information and verifying an identify.

11. A method for obtaining a prescription safety eyewear, said method comprising:
placing a template on a face;
aligning the face with a camera;
allowing an image of the face to be created;
adjusting a pupil alignment tool to align with pupils of the face;
adjusting a template alignment tool to align with the template;
selecting a one of a plurality of frames;
viewing the selected one superimposed on the image;
providing a prescription information; and
requesting a prescription safety eyewear to be manufactured from the selected of a plurality of frames.

12. The method of claim 11, further comprising the step of verifying a proper alignment of the image.

13. The method of claim 11, whereby the providing a prescription information step comprises submitting a prescription form displaying the prescription information to a scanner.

14. The method of claim 11, further comprising selecting a one of a plurality of lens options.

15. The method of claim 11, further comprising selecting a one of a plurality of shipping options.

16. The method of claim 11, further comprising the step of paying an excess of a cost of the prescription safety eyewear over an amount of benefits available.
